(19) United States
(12) Patent Application Publication
(10) Pub. No.: US 2003/0133982 A1
(43) Pub. Date: Jul. 17, 2003

(54) Zero-order sustained release dosage forms and method of making same

(76) Inventors: John M. Heimlich, Portage, MI (US); Loksidh D. Ganorkar, Kalamazoo, MI (US); Ernest J. Lee, Kalamazoo, MI (US); Robert M. Noack, Grand Rapids, MI (US); Ronald R. VerHage, Lawton, MI (US)

Correspondence Address: PHARMACIA CORPORATION, GLOBAL PATENT DEPARTMENT, POST OFFICE BOX 1027, ST. LOUIS, MO, 63006, US

(21) Appl. No.: 10/324,719

(22) Filed: Dec. 19, 2002

Related U.S. Application Data
(60) Provisional application No. 60/342,642, filed on Dec. 20, 2001.
(60) Provisional application No. 60/342,819, filed on Dec. 20, 2001.

(51) Int. Cl. A61K 9/26; A61K 9/14; A61K 31/445

(52) U.S. Cl. 424/469; 514/317; 514/49; 514/152; 514/262.1; 514/192; 514/561; 514/252.16; 514/253.08; 424/679; 514/211.07

(57) ABSTRACT

The present invention relates to zero-order sustained release solid dosage forms suitable for administration of a wide range of therapeutically active medicaments, especially those that are water-soluble, and to a process of making same. The solid dosage form comprises (a) a matrix core comprising ethylcellulose and the active agent and (b) a hydrophobic polymer coating encasing the entire matrix core.

This application claims the benefit of U.S. Provisional Application Serial No. 60/342,819, filed Dec. 20, 2001, and of U.S. Provisional Application Serial No. 06/342,642, filed Dec. 20, 2001.

### BACKGROUND OF THE INVENTION

1. Field of the Invention

The present invention relates to zero-order sustained release dosage forms suitable for administration of a wide range of therapeutically active medicaments, especially those that are water-soluble, and to a process of making same.

2. Description of the Related Art

There exists a significant need for a pharmaceutical delivery system which releases the active agent, especially a highly soluble agent, in zero-order release profile and over an extended period of time.

Sustained release dosage forms are well known in the art. As used herein, a sustained release dosage form refers to a drug dosage form which releases its drug content gradually and over an extended period of time after the drug makes contact with the environmental fluids. By “environmental fluid”, it is meant that the formulation is placed in an aqueous solution (e.g., in-vitro dissolution), in simulated gastric fluid (e.g., in accordance with the USP Basket Method (i.e., 37° C., 100 RPM, first hour 700 ml gastric fluid with or without enzymes at pH 1.2, then changed to 900 ml at pH 7.5), or in gastrointestinal fluid (in vivo). These dosage forms are desirable in the treatment of a number of diseases because the drug concentration is maintained in the body for longer periods of time, leading to reduction in the frequency of dosage. These dosage forms can be formulated into a variety of physical structures or forms, including tablets, lozenges, gelcaps, buccal patches, suspensions, solutions, gels, etc.

Most sustained release versions which are available in the market, however, do not have a zero-order release profile, that is, they do not produce uniform blood concentration levels for a prolonged period of time. Initially, the rate of drug release from most such formulations increases rapidly and is followed by a continuously declining rate of release at an exponential rate. This type of drug release is categorized as the first-order release.

Zero-order release dosage forms are also known in the art. The term “zero-order release dosage form” refers to a dosage form which releases its drug content at an uniform or nearly uniform rate independent of the drug concentration (in the dosage form) during a given period of release. Zero-order dosage forms generally provide maximum therapeutic value, while minimizing side effects.

Zero-order release dosage forms enable one to reduce dosing frequency compared to less sustained or more unevenly released dosage forms, thus improving the dosage compliance on the part of subjects. Zero-order release dosage forms also tend to maximize therapeutic value while minimizing the side effects. While zero-order sustained release dosage forms are known in the art, providing such a dosage form has proven to be difficult, particularly with highly soluble pharmaceutical agents at high drug load.

It has been found in the art that the high solubility in water of the active ingredient tends to generate a product that is susceptible to the phenomenon known as “dose dumping”. That is, release of the active ingredient is delayed for a time but once release begins the rate of release is very high. Most such systems available in the art are incapable of delivering the active agent with a zero-order profile for more than 12 hours.

Numerous matrix systems have been devised in an effort to achieve zero-order release of various active agents. Several controlled release systems comprising an active agent dispersed in an insoluble matrix encased by an insoluble coating, in which the active agent is exposed through an aperture in the coating, have been described. For example, EPA 259219 describes a ring shaped system in which the aperture is present in the center of the ring; U.S. Pat. No. 3,851,648 discloses a cylindrical device in which the aperture runs along the length of the cylinder and defines a cavity; European Pat. No. 0 656 204 describes a pharmaceutical tablet having lenticular form. The basis for these systems is that the surface area of exposed active agent continuously increases as dissolution proceeds, to compensate for the increased diffusion path between the aperture and the dissolving core.

U.S. Pat. No. 4,972,448 describes a coated right cylinder having an exposed circumferential strip.

U.S. Pat. No. 5,114,719 describes a polymeric device for extended delivery of small, water-soluble molecules in which the drug release is controlled by a specific manner of loading the biologically active molecules onto the core.

U.S. Pat. No. 4,838,177 describes a matrix system for releasing insoluble drugs into the system in granular form comprising a generally cylindrical core which is coated on one or both faces with an inert or insoluble polymeric material. The core is obtained by compression of the active substance and a swellable and gellable polymer or mixture of polymers. The release profile in this system is controlled by the high degree of swelling of the core.

U.S. Pat. No. 6,033,685 describes a layered tablet comprising a matrix layer and a barrier layer laminated to one or both faces of the matrix layer.

European Pat. No. 0 598 309 discloses a matrix system wherein the drug-containing matrix comprises swellable hydrophilic polymers.

Other matrix systems have been developed that do not require the presence of a hole or aperture in the polymer coating surrounding a matrix core. U.S. Pat. No. 4,919,939 discloses a tablet comprising a core matrix comprising a water soluble polymer, a hydroxypropylmethylcellulose gelling agent and a water soluble drug, with a water permeable ethyl cellulose polymer coating layer surrounding the core.

U.S. Pat. No. 4,892,742 discloses controlled release tablet formulations that include a core comprising a water soluble active ingredient in a water insoluble polymeric matrix, and a membrane coating comprising a rate controlling polymer. The only methods specifically disclosed therein for making such formulations involve the use of alcohol and other solvents were used in the process. Formulations of potassium chloride, produced as illustrated in the Examples section of the application released potassium chloride within 6 to 8 hours in a release rate study. (See Table I). This rate of release is too fast to make such formulations useful for once-a-day administration.

Many sustained release dosage forms known in the art are prepared using the technique called wet granulation. Wet granulation involves many steps, which could include: milling of drugs and excipients, mixing of the milled powders, preparation of binder solution, mixing of binder solution with powder mixture to form a wet mass, coarse screening of the wet mass, drying of moist granules, screening of dry granules, mixing of screened granules with lubricant and disintegrant, and tablet compression. Wet granulation is an expensive process because it requires many processing steps and involves considerable material handling equipment. Generally, free water and heat are inimical to the active ingredient. Wet granulation procedures involve water and/or heat.

What is needed is to provide a zero-order release oral dosage form with a sufficiently extended rate of release to permit it to be administered once-a-day. What is also needed is a method for making such dosage forms in the substantial absence of heat, free water, and other solvents in order to enhance the survival of any active ingredient incorporated into the formulation.

### SUMMARY OF INVENTION

It is an object of the present invention to provide a dosage form for water-soluble active agents which releases the active agent at zero-order for a period of at least 12 hours.

It is another object of the present invention to provide a zero-order sustained release solid dosage form that can be prepared easily on the production scale and that does not have the abovementioned disadvantages.

It is still another object of the present invention to provide a process for manufacturing a zero-order sustained release tablet which process is simple and allows manufacture of the tablet on a production scale.

It has been surprisingly and unexpectedly found that a delivery system provided for in the present invention is capable of delivering active agents having a wide range of solubility, particular those that are freely or very soluble in zero-order release profile for a period exceeding 12 hours and is capable of being manufactured on production scale by conventional dry granulation which does not use solvent or heat.

The present invention therefore relates to a zero-order sustained release solid solid dosage form comprising: (a) a matrix core comprising at least one water soluble active agent and intragranular ethylcellulose granulated and compressed together with extragranular cellulose, and (b) a film coating comprising a hydrophobic polymer, wherein the film coating completely encases the matrix core.

In a preferred embodiment, the hydrophobic polymer in the film coating is ethylcellulose.

In another preferred embodiment, the film coating further comprises a pore-former.

The present invention also relates to a process for manufacturing a zero-order sustained release tablet containing a water-soluble active agent, comprising the steps of: (a) preparing a first admixture comprising the active agent and intragranullar ethylcellulose; (b) granulating the first admixture in order to obtain a granular product; (c) preparing a second admixture comprising extragranular ethylcellulose; (d) preparing a third admixture comprising the granular product and the second admixture; (e) compressing the third admixture into a tablet core; and (f) applying a filming coating to the tablet core, said film coating comprising hydrophobic polymer.

### BRIEF DESCRIPTION OF THE DRAWINGS

FIG. 1 is a graph depicting the release profile in pH 6.8 phosphate buffer of (−)-S-3-(3-methylsulfonylphenyl)-N-n-propylpiperidine formulation prepared in accordance with the procedure set out in Example 1.

FIG. 2 is a graph showing the release profile in pH 6.8 phosphate buffer of three clindamycin HCl formulations, prepared with 4%, 6%, and 7% of 80/20 Surelease/HPMC coating, as described in Example 2.

FIG. 3 is a graph showing the bioabsorption profile after oral administration of single 600 mg. doses of each of three clindamycin HCl formulations, prepared for fast, medium, and slow extended zero-order release, and two 300 mg doses rapid of a commercial formulation of clindamycin HCl, Cleocin® (Pharmacia Corp.).

### DETAILED DESCRIPTION OF THE INVENTION

The present invention provides for a solid dosage form comprising

(a) a matrix core comprising at least one water soluble active agent and intragranular ethylcellulose granulated and compressed together with extragranular ethylcellulose, and

(b) a film coating comprising a hydrophobic polymer, wherein the film coating completely encases the matrix core.

In a particular embodiment the matrix core further comprises at least one pharmaceutically acceptable filler.

In another embodiment the matrix core further comprises at least one lubricant.

In a preferred embodiment the hydrophobic polymer in the film coating is ethylcellulose.

In yet another preferred embodiment the film coating further comprises at least one pore-former.

The term “intragranular”, as used herein, refers to a component of a formulation that is granulated with at least one other component of a formulation.

The term “extragranular”, as used herein, refers to a component of a formulation that is combined with intragranular components, after the intragranular components have been granulated.

The term “zero-order”, as used herein, refers to a uniform or nearly uniform active agent sustained release rate from a dosage form, independent of the concentration of the active agent in the dosage form during a given period of release.

The solid dosage forms of the present invention provide for zero-order or substantially zero-order, sustained release of the active agent. The active agent embedded in the matrix core diffuses through channels formed in the matrix and the film coating. The matrix core is prepared by conventional dry granulation methods without using a solvent. The film coating is applied using a conventional process known in the art. The coated tablets of the present invention have a dual advantage in allowing ease of manufacture and affording medicament release in a substantially linear fashion over an extended period of time.

No specialized geometry of the matrix core is necessary in the present invention. The matrix core may be in any shape known in the pharmaceutical industry and suitable for drug delivery, such as in spherical, cylindrical, or conical shape. In the case of cylindrical shape, it generally has flat, convex, or concave surfaces. Tablets are preferred.

One or more active agents may be combined in a single dosage form, depending on the chemical compatibility of the combined active ingredients and the ability to obtain the desired release rate from the solid dosage form for each active ingredient. Active agents suitable for the present inventions comprise any water soluble pharmacologically active compounds. Water soluble compounds are those molecules that require 30 or less parts of water (solvent) to dissolve one part of compound (solute). The United States Pharmacopoeia uses the descriptive terms “soluble” to mean from 10 to 30 parts solvent to dissolve one part solute, “freely soluble” to mean from 1 to 10 parts solvent to dissolve one part solute and “very soluble” to mean that less than one part solvent is needed to fully dissolve one part solute. For the purposes of this invention, all water soluble compounds are suitable for this drug delivery system. It is preferred that the active agents are freely and very soluble compounds. However, active agents that are either freely soluble or approach “freely soluble” are especially suitable for this invention. Examples of active agents suitable in the present invention include antihistamines, antibiotics, antituberculosis agents, cholinergic agents, antimuscarinics, sympathomimetics, sympatholytic agents, autonomic drugs, iron preparations, haemostatics, cardiac drugs, antihypertensive agents, vasodilators, non-steroidal antiinflammatory agents, opiate agonists, anticonvulsants, tranquilizers, stimulants, barbiturates, sedatives, expectorants, antiemetics, gastrointestinal drugs, heavy metal antagonists, antithyroid agents, genitourinary smooth muscle relaxants and vitamins. Examples of specific active agents include reboxetine, clindamycin, (−)-S-3-(3-methylsulfonylphenyl)-N-n-propylpiperidine, sumanirole, pramipexole, atenolol, propoxyphene, metformin, metoprolol, amitriptyline, ranitidine, fexofenadine, quinapril, sildenafil, tramadol, verapamil, gabapentin, potassium chloride, alendronate, bupropion, levofloxacin, doxycycline, venlafaxine, allopurinol, isosorbide mononitrate, fosonipril, propanolol, promethazine, captopril, fluvastatin, cimetidine, sumatriptan, nortriptyline, naproxen, calacyclovir, doxepin, amoxicillin, azithromycin, diltiazem, cefprozil, acyclovir, ciprofloxacin, losartan, and pharmaceutically acceptable salts of any of said active agent. It is preferred that the active agent is selected from the group consisting of reboxetine, clindamycin, (−)-S-3-(3-methylsulfonylphenyl)-N-n-propylpiperidine hydrochloride, sumanirole, pramipexole, and pharmaceutically acceptable salts of any of said active agent. The active agent is most preferably a form of clindamycin.

When the active agent is a form of clindamycin, it is suitably in any one of a number of bioavailable forms, including but not limited to clindamycin HCl, clindamycin phosphate, clindamycin palmitate, clindamycin free base (amorphous), and clindamycin crystalline free base. The clindamycin is preferably present in at least one form as clindamycin HCl, clindamycin phosphate, or clindamycin crystalline free base, more preferably as clindamycin HCl or as clindamycin crystalline free base, even more preferably as clindamycin HCl.

Crystalline clindamycin free base is disclosed in U.S. patent application Ser. No. 10/228,356, incorporated herein by reference. Crystalline clindamycin free base can be produced by either of the two alternative processes, illustrated in the above-referenced patent application. One illustrative process of preparing crystalline clindamycin free base involves forming the amorphous free base as a precipitate in aqueous medium followed by agitation to crystallize the free base from the precipitate. An illustrative example of the method involves first dissolving a salt of clindamycin, e.g., clindamycin hydrochloride in a solvent, preferably a polar solvent such as, for example, water. This if followed by adding an alkali material, i.e. a base, in an aqueous vehicle such as for example, a NaOH solution, such as, for example, preferably from about 0.01 to about 10 N NaOH solution, more preferably from about 0.1 to about 1 N NaOH, and more preferably about 0.5 N NaOH. This results in precipitation of the amorphous free base. The amorphous free base is then crystallized by agitation of the precipitate by, for example, by sonicating or manually shaking the precipitate, or by both sonicating and manually shaking the precipitate suspended in the aqueous medium. The crystallized free base is then preferably harvested by centrifugation, followed by removal of the liquid portion. The crystallized free base is preferably washed in at least one washing step involving adding a wash solution, sonicating, shaking, centrifuging and removing the wash solution from the crystalline material. The wash solution is preferably aqueous, more preferably water.

In an alternate method, crystalline clindamycin free base can be produced by a slow addition of a clindamycin salt, such as clindamycin hydrochloride, dissolved in a polar solvent such as water to an aqueous alkaline solution containing a water-soluble organic substance, preferably an alcohol co-solvent. The aqueous solution containing an alkali with an alcohol co-solvent is prepared by adding the alkali, i.e. base, in an aqueous vehicle such as, for example, a NaOH solution. The NaOH solution can be, for example, preferably from about 0.01 to about 10 N NaOH solution, more preferably from about 0.1 to about 1 N NaOH, and more preferably about 0.5 N NaOH. The alcohol co-solvent is present, preferably in an amount of from about 2% to about 20%, more preferably from about 5% to about 10%. Any of a number of alcohols that are readily miscible with water can be used, preferably, methanol, ethanol, n-propanol, t-butanol and the like. Typically alcohols of higher molecular weight are less soluble in water and less preferred. Diols such as 1,2, ethanediol (ethylene glycol), 1,2 propanediol (propylene glycol) and 1,2 butanediol and triols such as 1,2,3 propantriol (glycerol) and the like can also be used as co-solvent. It is also possible to use an aqueous solution of a water-soluble organic substance such as, for example, sodium acetate.

An aqueous solution of a clindamycin salt, such as, for example clindamycin hydrochloride is prepared and slowly added to the alkali solution with alcohol co-solvent, preferably over a period of from about 15 minutes to about 4 hours, more preferably from about 30 minutes to about 2 hours and most preferably from about 45 minutes to 75 minutes. Crystallization is allowed to proceed for I to 24 hours and the crystalline free base material is isolated by filtration, centrifugation and decanting or the like. In a preferred variation of this method, the clindamycin hydrochloride solution is added in a multi-phase infusion schedule such as, for example, a first phase of slow infusion over about one hour, followed by a faster infusion phase over about 30 min and concluding with slow infusion phase over about one hour.

The material obtained by either of the methods above is isolated and dried, for example, under a stream of humidified nitrogen. The dry material can be further processed such as by grinding to produce a dry powder.

More than one active agent or form of a single active agent are suitably used in the solid dosage forms of the present invention. Selection of the form of active agent or combination of forms to include in any given solid dosage form of the present invention depends, at least in part, upon the desired release properties and the solubility of each form of active agent. For example, clindamycin HCl is highly soluble in water, while clindamycin crystalline free base is considerably less soluble. Amorphous clindamycin free base is the least soluble of all the forms of clindamycin listed above. By using two or more different forms of clindamycin in a composition of the present invention, each of which has a different solubility in water, one can vary the release rate of clindamycin after oral administration. However, release rates can also be controlled using various excipients, polymers, and matricies, such as are described below. Thus, it is contemplated but not necessary for the formulations of the present invention to comprise more than one form of clindamycin.

The amount of active agent in the matrix core may be adjusted based on a variety of parameters such as physical-chemical properties of the active agents, solubility, required therapeutic dose levels, half life in blood, and so on. Generally, the active agent content is from about 1% to about 85%, but is preferably from about 5% to about 75%, more preferably from about 20% to about 70%, and even more preferably from about 50% to 70%, wherein the weight percentage is based on the total weight of the matrix core.

The matrix core preferably contains at least a therapeutically effective amount of the active agent. It will be understood that a therapeutically effective amount of an active agent for any given subject is dependent inter alia on the body weight of the subject. Where the subject is a child or a small animal (e.g., a dog), for example, the amount of clindamycin required to provide blood serum concentrations consistent with therapeutic effectiveness is relatively less than the amount required to provide comparable blood serum concentrations in an adult human or a large animal.

Ethylcellulose suitable for use in the matrix core in the present invention can be a standard type viscosity grade that contains 46.5% or more ethoxy groups or a medium type viscosity grade that contains less than 46.5% ethoxy groups. Example of a suitable grade of ethylcellulose is available from Dow Chemical Co. of Midland, Mich. under the trade name ETHOCEL® and exhibits a viscosity in a 5% solution measured at 25° C. in solvent of 80% toluene and 20% alcohol of about 6-100 cps, preferably 9-11 cps and most preferably about 10 cps. The particle size of the ethylcellulose ranges from 3-60 μm with 3-15 μm being most preferred. The same type of ethylcellulose is preferably used as the intragranular and extragranular ethylcellulose in the matrix core of the present formulations. Certain relative amounts of intragranular ethylcellulose and extragranular ethylcellulose are preferred in the matrix core, in view of ease of manufacture and other factors described, herein below.

The total content of ethylcellulose in the matrix core is preferably from about 15% to about 99%, more preferably from about 20% to about 45%, more preferably from 20% to about 35%, and even more preferably from 20% to about 30%, by weight, relative of the total weight of the matrix core.

Through selection and combination of excipients, compositions can be provided exhibiting improved performance with respect to, among other properties, efficacy, bioavailability, clearance time, stability, compatibility of drug and excipients, safety, dissolution profile, disintegration profile and/or other pharmacokinetic, chemical and/or physical properties. Where the composition is formulated as a tablet, the combination of excipients selected provides tablets that can exhibit improvement, among other properties, in dissolution profile, hardness, crushing strength, and/or friability.

The matrix core of the solid dosage forms of the present invention may include at least one pharmaceutically acceptable filler as an excipient. The term “fillers” used herein means the fillers which are used for ordinary pharmaceutical production, and includes excipients which facilitate the compression of powdery materials and give the solid dosage forms strength. The following are examples of suitable fillers for use in the matrix core of the present invention: microcrystalline cellulose, sodium citrate, dicalcium phosphate, colloidal silicon dioxide, starches, lactose, sucrose, glucose, mannitol, and silicic acid, alginates, gelatin, polyvinylpyrrolidinone, and acacia, with microcrystalline cellulose being preferred. Of the different types of microcrystalline cellulose available on the market, Avicel-PH-101 and, Avicel-PH-102 (available from FMC Corporation, American Viscose Division, Avicel Sales, Marcus Hook, Pa., U.S.A.) are preferred. The filler may be present in an amount up to about 50% of the total weight of the uncoated matrix core. The content of the filler in the matrix core may be increased or decreased based on various factors such as active agent load, active agent solubility, and desired release profile. Generally the content of the filler is in reverse order with the load of the active agent. It is preferred that the filler is present in an amount up to about 20% of the total weight of the matrix core for very high loads of the active agent and from about 30% to 50% of the total weight of the matrix core for very low loads of the active agent.

The matrix core of the solid dosage form of the present invention may further comprise at least one pharmaceutically acceptable lubricant (including anti-adherents and/or glidants) as an excipient. The term “lubricant” as used in this description includes excipients that reduce inter-particle friction inside the solid dosage form, reducing the reaction forces appearing on the walls of the matrix. Suitable lubricants include, either individually or in combination, glyceryl behapate (e.g., Compritol™ 888); stearic acid and salts thereof, including magnesium, calcium and sodium stearates; hydrogenated vegetable oils (e.g., Sterotex™); colloidal silica; talc; waxes; boric acid; sodium benzoate; sodium acetate; sodium fumarate; DL-leucine; PEG (e.g., Carbowax™ 4000 and Carbowax™ 6000); sodium oleate; sodium lauryl sulfate; and magnesium lauryl sulfate. The lubricant is more preferably selected from the group consisting of stearic acid salts such as calcium stearate and magnesium stearate, stearic acid, stearate family, sodium stearyl fumarate, solid polyethylene glycols, sodium lauryl sulfate, and mixtures thereof. Magnesium stearate is a particularly preferred lubricant. When present, the amount of lubricant present in the matrix core is preferably from about 0.1% to about 3.0%, more preferably from about 0.2% to about 2.0%, and most preferably from 0.25% to about 1.0%, by weight, relative of the total weight of the uncoated matrix core.

The solid dosage forms of the present invention optionally comprise one or more pharmaceutically acceptable diluents as excipients. Suitable diluents illustratively include, either individually or in combination, lactose, including anhydrous lactose and lactose monohydrate; starches, including directly compressible starch and hydrolyzed starches (e.g., Celutab™ and Emdex™); mannitol; sorbitol; xylitol; dextrose (e.g., Cerelose™ 2000) and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, amorphous cellulose (e.g., Rexcel™) and powdered cellulose; calcium carbonate; glycine; bentonite; and polyvinylpyrrolidone. Such diluents, if present, constitute in total about 5% to about 99%, preferably about 10% to about 85%, and more preferably about 20% to about 80%, of the total weight of the composition. The diluent or diluents selected preferably exhibit suitable flow properties and, where tablets are desired, compressibility.

Microcrystalline cellulose is a preferred diluent, particularly when the active agent is clindamycin. Microcrystalline cellulose is chemically compatible with clindamycin. The use of extragranular microcrystalline cellulose (that is, microcrystalline cellulose added to a granulated composition) can be used to improve hardness (for tablets) and/or disintegration time. It typically provides compositions having suitable release rates of clindamycin, stability, flowability, and/or drying properties at a relatively low diluent cost. It provides a high density substrate that aids densification during granulation and therefore improves blend flow properties.

Through selection and combination of excipients, compositions can be provided exhibiting improved performance with respect to, among other properties, efficacy, bioavailability, clearance time, stability, compatibility of drug and excipients, safety, dissolution profile, disintegration profile and/or other pharmacokinetic, chemical and/or physical properties. Where the composition is formulated as a tablet, the combination of excipients selected provides tablets that can exhibit improvement, among other properties, in dissolution profile, hardness, crushing strength, and/or friability.

The solid dosage forms of the present invention optionally comprise one or more pharmaceutically acceptable binding agents or adhesives as excipients, particularly for tablet formulations. Such binding agents and adhesives preferably impart sufficient cohesion to the powder being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; sucrose; gelatin; glucose; starches such as, but not limited to, pregelatinized starches (e.g., National™ 1511 and National™ 1500); celluloses such as, but not limited to, microcrystalline cellulose, methylcellulose and carmellose sodium (e.g., Tylose™); alginic acid and salts of alginic acid; magnesium aluminum silicate; PEG; guar gum; polysaccharide acids; bentonites; povidone, for example povidone K-15, K-30 and K-29/32; polymethacrylates; HPMC; hydroxypropylcellulose (e.g., Klucel™); and ethylcellulose (e.g., Ethocel™). Such binding agents and/or adhesives, if present, constitute in total about 0.5% to about 25%, preferably about 0.75% to about 15%, and more preferably about 1% to about 10%, of the total weight of the solid dosage form.

Solid dosage forms of the present invention optionally comprise one or more pharmaceutically acceptable wetting agents as excipients. Non-limiting examples of surfactants that can be used as wetting agents in compositions of the invention include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene (8) caprylic/capric mono- and diglycerides (e.g., Labrasol™ of Gattefossé), polyoxyethylene (35) castor oil and polyoxyethylene (40) hydrogenated castor oil; polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefossé), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Such wetting agents, if present, constitute in total about 0.25% to about 15%, preferably about 0.4% to about 10%, and more preferably about 0.5% to about 5%, of the total weight of the solid dosage form.

Suitable anti-adherents include talc, cornstarch, DL-leucine, sodium lauryl sulfate, colloidal silica, and metallic stearates. Talc is a preferred anti-adherent or glidant used, for example, to reduce formulation sticking to equipment surfaces and also to reduce static in the blend. Talc or colloidal silica, if present, constitute about 0.1% to about 10%, more preferably about 0.25% to about 5%, and still more preferably about 0.5% to about 2%, of the total weight of the composition.

Other excipients such as colorants, flavors and sweeteners are known in the pharmaceutical art and can be used in compositions of the present invention. Tablets can be coated, for example with an enteric coating, or uncoated. Compositions of the invention can further comprise, for example, buffering agents.

The solid dosage forms of the present invention are granulated prior to compression. Granulation, among other effects, densifies milled compositions resulting in improved flow properties, improved compression characteristics and easier metering or weight dispensing of the compositions for encapsulation or tableting. The secondary particle size resulting from granulation (i.e., granule size) is not narrowly critical, it being important only that the average granule size preferably is such as to allow for convenient handling and processing and, for tablets, to permit the formation of a directly compressible mixture that forms pharmaceutically acceptable tablets.

The film coating of the solid dosage form of the present invention comprises a non-swelling hydrophobic polymer. The film coating completely encases the entire matrix core and further controls the release of the active agent. The non-swelling hydrophobic polymers suitable for use in the coating in the present invention include, but are not limited to, water insoluble material such as a wax or a wax-like substance, fatty alcohols, shellac, zein, hydrogenated vegetable oils, water insoluble celluloses such as ethylcellulose, cellulose acetate, polymers of acrylic and/or methacrylic acid, and any other slowly digestible or dispersible solids known in the art. Ethylcellulose is a preferred hydrophobic polymer for use in the film coating.

Ethylcellulose suitable for use in the film coating in the present invention can be a standard ethylcellulose dispersion that contains ethylcellulose, a suitable plasticizer, and stabilizers. An example of a suitable grade of ethylcellulose dispersion is available from Colorcon, Inc. of West Point, Pa., under the tradename SURELEASE®, which contains approximately 25% solids by weight, and coalesces to form a suitable film when applied to tablets.

Suitable hydrophobic acrylic polymer used in the coatings of the present invention comprises copolymers of acrylic and methacrylic acid esters with a low content of quaternary ammonium groups. Such copolymers are often referred to as ammonio methacrylate copolymers, and are commercially available from Rohm Pharma AG, e.g., under the trade name Eudragit®. Ammonio methacrylate copolymers are described in NF XVII as fully polymerized copolymers of acrylic and methacrylic acid esters with a low content of quaternary ammonium groups.

In certain preferred embodiments of the present invention, the acrylic coating is derived from a mixture of two acrylic resin lacquers used in the form of aqueous dispersions, commercially available from Rohm Pharma under the trade name Eudragit® RL 30 D and Eudragit® RS 30 D, respectively. Eudragit® RL 30 D and Eudragit® RS 30 D are copolymers of acrylic and methacrylic esters with a low content of quaternary ammonium groups, the molar ratio of ammonium groups to the remaining neutral (meth)acrylic esters being 1:20 in Eudragit® RL 30 D and 1:40 in Eudragit® RS 30 D. The mean molecular weight is about 150,000. The code designations refer to the permeability properties of these agents, RL for high permeability and RS for low permeability. Eudragit® RL/RS mixtures are insoluble in water and in digestive fluids. However, coatings formed from the same are swellable and permeable in aqueous solutions and digestive fluids. The Eudragit® RL/RS dispersions of the present invention may be mixed together in any desired ratio in order to ultimately obtain a controlled release formulation having a desirable dissolution profile. Desirable controlled release formulations may be obtained, for instance, from a retardant coating derived from 100% Eudragit® RL, 50% Eudragit® RL and 50% Eudragit® RS, and 10% Eudragit® RL: Eudragit®90% RS, and 100% Eudragit® RS.

The film coating is preferably applied to the matrix core to achieve a weight gain level from about 1% to about 33%, preferably from about 3% to about 15%, more preferably from 3% to about 12%, and even more preferably from about 5% to about 10%. However, the film coat may be lesser or greater depending upon many factors such as the physical properties of the soluble drug(s) included in the formulation, the desired release rate, and the desired drug load.

The film coating may further comprise one or more pore formers. The addition of the pore-former help further adjust the release of the active agent from the controlled release solid dosage form of the present invention. The term “pore-former” include materials that can be dissolved, extracted or leached from the coating in the environment of use. Upon exposure to fluids in the environment of use, the pore-formers are, e.g., dissolved, and channels and pores are formed that fill with the environmental fluid.

The pore-formers can be inorganic or organic and can be solids and liquids. The pore-forming solids have a size, e.g., of about 0.1 to 200 microns and they include alkali metal salts such as lithium carbonate, sodium chloride, sodium bromide, potassium chloride, potassium sulfate, potassium phosphate, sodium acetate, sodium citrate, suitable calcium salts, and the like.

Pore formers can be water-soluble hydrophilic polymers. Examples of suitable hydrophilic polymers include hydroxypropyl methylcellulose, cellulose ethers, protein-derived materials, polyvinylpyrrolidone, cross-linked polyvinylpyrrolidone, polyethylene oxide, polyethylene glycol, water-soluble polydextrose, saccharides and polysaccharides, such as pullulan, dextran, sucrose, glucose, fructose, mannitol, lactose, mannose, galactose, sorbitol and the like. Of these hydrophilic polymer pore formers, hydroxypropyl methylcellulose is particularly preferred. A suitable form of hydroxypropyl methylcellulose is that having a viscosity in the range 3 to 100 cps at 20° C. (U.S. National Formulary XIII), and preferably a viscosity of approximately 3 cps at 20° C.

The amount of pore-former included in the film coatings of the present invention may be up to about 50%, preferably from about 10% to about 50%, more preferably from 15 to about 50%, and even more preferably from about 20 to 50%, by weight relative to the total weight of the film coating.

The relative amounts of pore former and ethylcellulose in the film coating can be varied to adjust the rate of release, with larger proportions of pore former resulting in faster release rates compared to smaller proportions of the same. In a preferred embodiment, the film coating comprises about 50% to about 100% by weight of ethylcellulose and about 50% to 0% by weight of hydroxypropyl methylcellulose. In another preferred embodiment, the film coating comprises about 50% to about 90% of ethylcellulose and about 50% to about 10% of hydroxypropyl methylcellulose. In a more preferred embodiment, the film coating comprises about 50% to 85% of ethylcellulose and about 50% to about 15% of hydroxypropyl methylcellulose. In a particularly preferred embodiment, the film coat comprises most preferably about 50% to about 80% of ethylcellulose and about 50% to about 20% of hydroxypropyl methylcellulose.

The solid dosage form of the present invention preferably releases the active agent contained therein at a zero-order rate for a period of at least 8 hours after oral administration, more preferably for a period of at least 12 hours after oral administration, even more preferably for a period of at least 18 hours after oral administration.

The solid dosage form of the present invention is preferably a coated tablet prepared using conventional techniques known in the art.

The matrix core is prepared by conventional dry granulation technologies known in the art. A first admixture comprising the active agent and a first portion of ethylcellulose, the intragranular ethylcellulose is dry admixed. If a lubricant is used, the first admixture further comprises a portion of the lubricant. The first admixture is then granulated to form a granular product. The granulated product is then combined with a second admixture comprising the remaining portion of ethylcellulose to be included in the matrix core, the extragranular ethylcellulose. The second admixture further comprises a lubricant or pharmaceutically acceptable filler or both, when either or both additional component is to be included in the matrix core. The granular product prepared from the first admixture is then admixed with the second admixture to form a third admixture. The third admixture is then compressed to form the matrix core. The compression step may be done with a conventional tabletting machine.

Finally, the polymeric film coating is applied to the matrix core in a coating pan or by conventional spraying techniques. The polymeric film coating preferably comprises ethylcellulose.

In order to facilitate manufacture of the solid dosage form of the present invention, and to maintain the release profile of the present solid dosage form, it is necessary that the ethylcellulose in the matrix core have both a portion inside the granule (the intragranular ethylcellulose) and a portion exterior to the granule (the extragranular ethylcellulose). The extragranular ethylcellulose is preferably about 3% to about 15%, more preferably from about 5% to about 12%, and even more preferably from about 8% to about 10% of the matrix core weight.

The total amount of ethylcellulose in the matrix core is preferably about 15% to about 99%, by weight, relative to total weight of the matrix core, more preferably about 20% to about 45%, by weight, relative to the total weight of the matrix core, even more preferably about 20% to about 35%, by weight, relative to total weight of the matrix core.

The solid dosage forms produced by the process of the present invention, described immediately above, are extremely durable, and do not appreciably erode during the dissolution process. The solid dosage forms preferably maintain their integrity for an extended period of time during dissolution, and slowly release the drug in a zero-order or in a substantially zero-order fashion for a period of at least 12 hours. The solid dosage forms also preferably do not swell appreciably in the dissolution media, which allows for the tablets to retain a functional coat without rupture for an extended period of the dissolution process. The functional coat further controls the release of the drug from the solid dosage form.

The characteristics of any particular solid dosage form and process for producing the solid dosage form of the present invention can be adjusted to accommodate a variety of drugs with different characteristics to produce any given desired release rate. The solid dosage forms of the present invention are particularly useful for delivery of active agents in the form of highly soluble drugs that require a high drug load. The release of such drugs can be slowed to a desired release rate by modifying the system components.

Without further elaboration, it is believed that one skilled in the art can, using the preceding description, practice the present invention to its fullest extent. The following detailed examples describe how to prepare the various solid dosage forms and/or perform the various processes of the invention and are to be construed as merely illustrative, and not limitations of the preceding disclosure in any way whatsoever. Those skilled in the art will promptly recognize appropriate variations from the procedures both as to ingredients of the solid dosage forms and the process of making same.

### EXAMPLES

### Example 1

| | | |
|---|---|---|
| Amt (mg) | Wt. % | Component |

Intra-granular Ingredients

| | | |
|---|---|---|
| 75* | 42.9 | (−)-S-3-(3-methylsulfonylphenyl)-N-n- |
| | | propylpiperidine BULK DRUG (FBE) |
| 43.75 | 25.0 | Ethocel Std 10 Prem. FP Ethylcellulose |
| 0.4375 | 0.25 | Magnesium Stearate NF Powder Food Grade-V- |
| | | Bolted |

Extra-granular Ingredients

| | | |
|---|---|---|
| 28.43** | 16.2 | Microcrystalline Cellulose NF Coarse powder |
| 26.25 | 15.0 | Ethocel Std 10 Prem. FP Ethylcellulose |
| 0.7 | 0.4 | Colloidal Silicon Dioxide NF |
| 0.4375 | 0.25 | Magnesium Stearate NF Powder Food Grade-V- |
| | | Bolted |
| 175.0 | | Total Tablet weight |

Coating (10% weight gain)

| | |
|---|---|
| 2.625 | Hydroxypropyl methylcellulose |
| 14.875 | Surelease |
| 192.5 | Total System Weight |

*To be adjusted for API potency.
**The quantity of Microcrystalline Cellulose per tablet will be adjusted (q.s.'d) such that the total of the API + Microcrystalline Cellulose = 103.43 mg.

The following procedure was used to prepare coated tablets according to the formula set forth above:

Granular Phase

1. All intragranular ingredients with the exception of the Magnesium Stearate NF Powder Food Grade-V-Bolted were weighed.

2. The ingredients weighed in step 1 were screened using a 30 mesh hand screen.

3. The screened ingredients were dry mixed in a suitable blender (a PK blender) for 7 minutes.

4. The intragranular magnesium stearate was then weighed and manually blended with a portion of the step 3 mixture.

5. The ingredients mixed in sep 4 were placed into the mixing container with the remaining ingredients from step 4, and mixed for an additional 3 minutes.

6. The resulting mixture was run through a roller compactor to achieve a suitable ribbon.

7. The roller compacted ribbon was further processed in a second milling step, using a suitable mill (a Comil mill).

8. Material remaining after the first milling step was separated by sieving, using 20 and 80 mesh screens. All material retained on the 80 mesh screen was separated and retained as final granulation material. Material that passed through all screens was passed through the roller compactor for another granulation step. Material retained on 20 mesh screen was subjected to the second milling step (step 7).

9. Steps 6-8 were repeated three times or until acceptable yield is obtained.

10. The final milled material was sized by passing the granules through a 16 mesh screen. The material that passed through the 16 mesh screen was placed on an 80 mesh screen. The material that was retained on the 80 mesh screen was used for further processing.

Extragranular Phase:

11. All extragranular ingredients, with the exception of the Magnesium Stearate NF Powder Food Grade-V-Bolted, were weighed. The weight of the extragranular ingredients was adjusted to match the yield of intragranular material in step 10, above.

12. The materials from step 11 were screened using a 30 mesh hand screen.

13. The screened extragranular ingredients from step 12 were dry mixed with the final milled intragranular material from step 10, in a suitable blender (a PK blender) for 7 minutes.

14. The extragranular magnesium stearate was weighed and manually blended with a portion of the step 13 mixture.

15. The premixed ingredients from step 14 were placed back into the blender containing the remainder of the extragranular ingredients from step 13, and mixed for an additional 3 minutes.

16. The tablets were compressed using a 0.540×0.230″ capsule shaped tooling to obtain tablets of suitable hardness.

17. The resulting tablets were coated using an 80/20 mix of HPMC/Surelease to achieve the targeted weight gain.

18. The resulting coated tablets were tested in a pH 6.8 phosphate buffer, according to the procedure described in the US Pharmacopeia XXIII, Apparatus 1 at 100 rpm, with n=3.

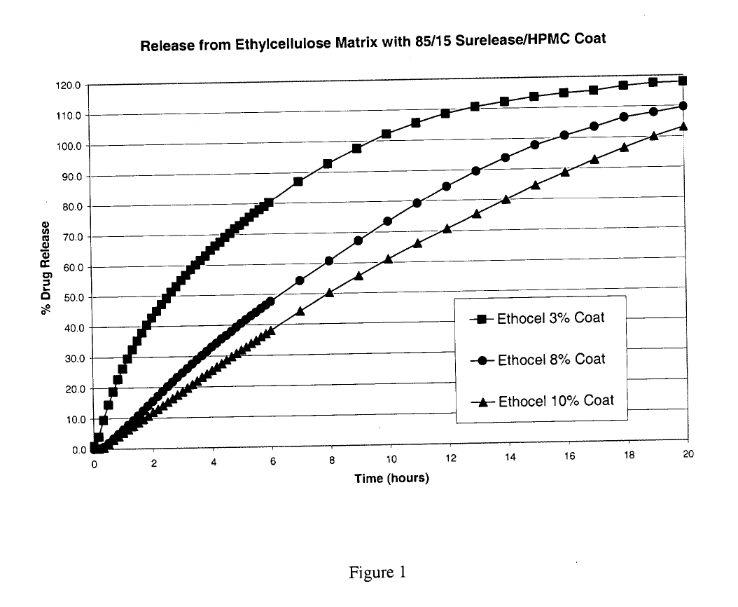

FIG. 1 shows the release profile of (−)-S-3-(3-methylsulfonylphenyl)-N-n-propylpiperidine tablet with the above formulation, prepared and tested in a pH 6.8 phosphate buffer.

### Example 2

| | | |
|---|---|---|
| Amt. (mg) | Wt. % | Component |

Intra-granular Ingredients

| | | |
|---|---|---|
| 600* | 76.44 | Clindamycin HCl |
| 162.7 | 18.08 | Ethocel Std 10 Prem. FP Ethylcellulose |
| 2.2 | 0.25 | Magnesium Stearate NF Powder Food Grade-V- |
| | | Bolted |

Extra-granular Ingredients

| | | |
|---|---|---|
| 44.89 | 4.99 | Ethocel Std 10 Prem. FP Ethylcellulose |
| 2.24 | 0.25 | Magnesium Stearate NF Powder Food Grade-V- |
| | | Bolted |
| 900.12 | 100 | Total Tablet weight |

Coating (6% weight gain)

| | |
|---|---|
| 10.8 | Hydroxypropyl Methylcellulose |
| 43.2 | Surelease ® Grade E-7-19010 (Colorcon, Inc.) |
| 954.1 | Total System Weight |

*To be adjusted for API potency.

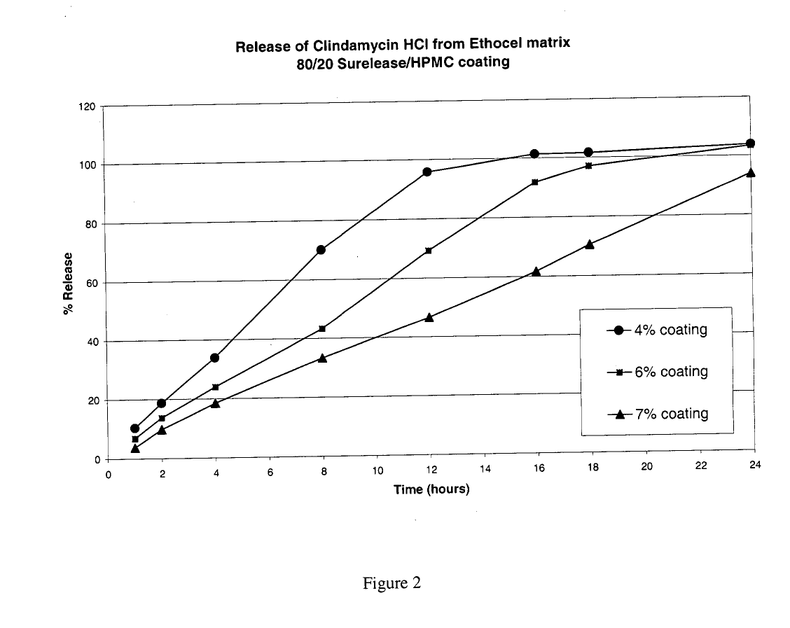

FIG. 2 shows the release profile of the 600 mg Clindamycin HCl tablets, prepared as described immediately above, with a pH 6.8 phosphate buffer.

### Example 3

Three sets of coated tablets of clindamycin HCl were prepared, as described in Example 1, above, using the same formula as in Example 2.The three test formulations described above were designed for three different rates of release of 600 mg of clindamycin HCl, fast (6 hour release), medium (9 hour release), and slow release (11 hour release). Bioavailability of clindamycin HCl from each of the above-cited formulations was compared to bioavailability of clindamycin HCl from two successive 300 mg doses of an immediate release commercial formulation of clindamycin, Cleocin Capsules, where administration of the Cleocin doses were separated by 12 hours. All doses were administered orally to human volunteers. 20 healthy adult volunteers were included in the study.

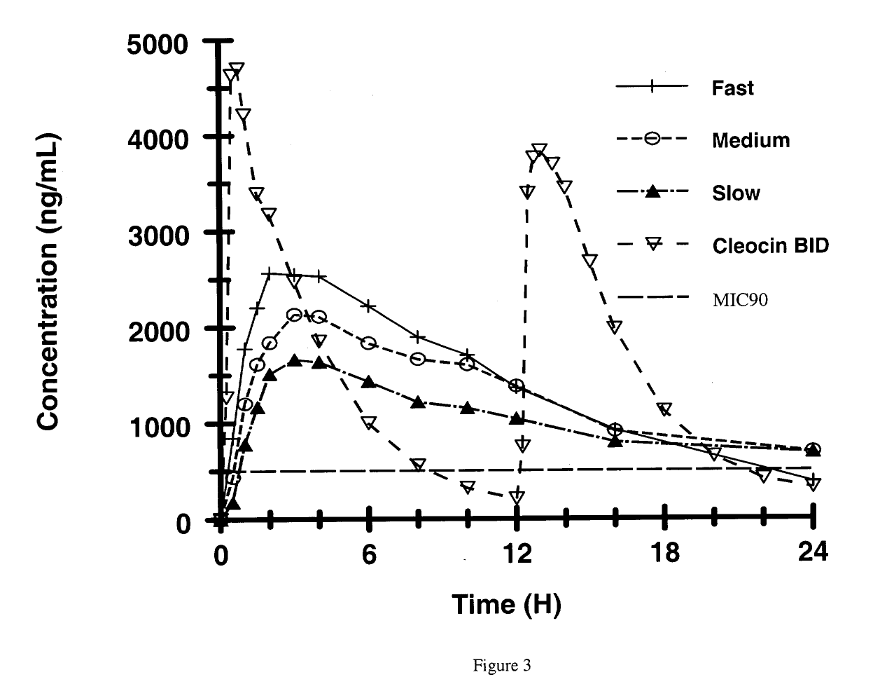

The study results are shown in FIG. 3, below. Bioavailable clindamycin HCl was found in the bloodstreams of all volunteers administered the extended release formulations, even 16 hours after administration. By comparison, the amount of bioavailable clindamycin HCl from the immediate release formulations dropped off dramatically after oral administration, and dropped below MIC90 about 8 hours after administration.

What is claimed is:

1. A solid dosage form, comprising
a matrix core comprising intragranular ethylcellulose and a water soluble active agent granulated and compressed together with extragranular ethylcellulose, and
a film coating comprising a hydrophobic polymer, wherein the film coating completely encases the matrix core.

2. The solid dosage form of claim 1 wherein the active agent is released at a zero-order rate for a period of at least 8 hours after oral administration to a subject.

3. The solid dosage form of claim 1, wherein the solid dosage form is a tablet.

4. The solid dosage form of claim, 1, wherein the active agent is selected from the group consisting of reboxetine, clindamycin, (−)-S-3-(3-methylsulfonylphenyl)-N-n-propylpiperidine, sumanirole, pramipexole, atenolol, propoxyphene, metformin, metoprolol, amitriptyline, ranitidine, fexofenadine, quinapril, sildenafil, tramadol, verapamil, gabapentin, potassium chloride, alendronate, bupropion, levofloxacin, doxycycline, venlafaxine, allopurinol, isosorbide mononitrate, fosonipril, propanolol, promethazine, captopril, fluvastatin, cimetidine, sumatriptan, nortriptyline, naproxen, calacyclovir, doxepin, amoxicillin, azithromycin, diltiazem, cefprozil, acyclovir, ciprofloxacin, losartan, and a pharmaceutically acceptable salt of any said active agent.

5. The solid dosage form of claim 1, wherein the active agent is selected from the group consisting of reboxetine, clindamycin, (−)-S-3-(3-methylsulfonylphenyl)-N-n-propylpiperidine hydrochloride, sumanirole, pramipexole, and a pharmaceutically acceptable salt of any of said active agent.

6. The solid dosage form of claim 1, wherein the intragranular and extragranular ethylcellulose together are present in an amount from about 15% to about 99%, by weight of the matrix core.

7. The solid dosage form of claim 1, wherein the matrix core further comprises a filler.

8. The solid dosage form of claim 7, wherein the filler is selected from the group consisting of microcrystalline cellulose, sodium citrate, dicalcium phosphate, colloidal silicon dioxide, starches, lactose, sucrose, glucose, mannitol, and silicic acid, alginates, gelatin, polyvinylpyrrolidinone, and acacia.

9. The solid dosage form of claim 7, wherein the filler is microcrystalline cellulose.

10. The solid dosage form of claim 7, wherein the amount of the filler is up to 50%, by weight, of the matrix core.

11. The solid dosage form of claim 1 wherein the matrix core further comprises a lubricant.

12. The solid dosage form of claim 11 wherein the lubricant is selected from the group consisting of stearic acid salts, stearic acid, stearate family, sodium stearyl fumarate, solid polyethylene glycols, sodium lauryl sulfate, and mixtures thereof.

13. The solid dosage form of claim 11 wherein the lubricant is magnesium stearate.

14. The solid dosage form of claim 11 wherein the amount of the lubricant is from about 0.1% to about 3.0%, by weight, of the matrix core.

15. The solid dosage form of claim 1 wherein the film coating comprises from about 1% to about 33%, by weight, relative to the weight of the matrix core.

16. The solid dosage form of claim 1, wherein the hydrophobic polymer of the film coating is selected from the group consisting of wax, wax-like substance, fatty alcohols, shellac, zein, hydrogenated vegetable oils, water insoluble celluloses, cellulose acetate, polymers of acrylic acid, and polymers of methacrylic acid.

17. The solid dosage form of claim 1, wherein the hydrophobic polymer comprises ethylcellulose.

18. The solid dosage form of claim 17, wherein in the ethylcellulose is about 50% to about 95% by weight of the film coating, and the film coating further comprises about 5% to about 50% by weight of hydroxypropyl methylcellulose.

19. The solid dosage form of claim 1, wherein the film coating further comprises a pore former.

20. The solid dosage form of claim 19, wherein the pore former is selected from the group consisting of lithium carbonate, sodium chloride, sodium bromide, potassium chloride, potassium sulfate, potassium phosphate, sodium acetate, sodium citrate, hydroxypropyl methylcellulose, cellulose ethers and protein-derived materials, polyvinylpyrrolidone, cross-linked polyvinylpyrrolidone, polyethylene oxide, polyethylene glycol, pullulan, dextran, sucrose, glucose, fructose, mannitol, lactose, mannose, galactose, and sorbitol.

21. The solid dosage form of claim 19, wherein the pore former is hydroxypropyl methylcellulose.

22. The solid dosage form of claim 19 wherein the amount of the pore former in the film coating is up to about 50%, by weight, of the film coating.

23. The solid dosage form of claim 1 wherein the active agent is in an amount from 1% to 85%, by weight, of the matrix core.

24. A solid dosage form, comprising:
a matrix core comprising, by weight relative of total weight of the matrix core,
about 20% to about 45% of ethylcellulose, up to about 50% of microcrystalline cellulose, and
about 40% to about 80% of a water soluble active agent, wherein the ethylcellulose, microcrystalline cellulose, and active agent are granulated and. compressed together; and
a film coating comprising, by weight relative of total weight of the film coating,
about 50% to about 95% of ethylcellulose, and
about 5% to about 50% of hydroxypropyl methylcellulose,
wherein the film coating completely encases the matrix core, and wherein the film coating comprises about 3% to about 15%, by weight, relative to the weight of the matrix core.

25. The solid dosage form of claim 24 wherein the active agent is selected from the group consisting of reboxetine, clindamycin, (−)-S-3-(3-methylsulfonylphenyl)-N-n-propylpiperidine, sumanirole, pramipexole, and pharmaceutically acceptable salt of any said active agents.

26. A process of making a solid dosage form,:
a. preparing a first, admixture comprising the active agent and intragranular ethylcellulose;
b. granulating the first admixture in order to obtain a granular product;
c. preparing a second admixture comprising extragranular ethylcellulose;
d. preparing a third admixture comprising the granular product and the second admixture;
e. compressing the third admixture to form a matrix core; and
f. applying a film coating to the matrix core, the film coating comprising hydrophobic polymer.

27. The process of claim 26 wherein the extragranular ethylcellulose is present in the second admixture in an amount from about 3% to about 15%, by weight, relative to the weight of the matrix core.

28. The process of claim 26 wherein the active agent is selected from the group consisting of reboxetine, clindamycin, (−)-S-3-(3-methylsulfonylphenyl)-N-n-propylpiperidine, sumanirole, pramipexole, atenolol, propoxyphene, metformin, metoprolol, amitriptyline, ranitidine, fexofenadine, quinapril, sildenafil, tramadol, verapamil, gabapentin, potassium chloride, alendronate, bupropion, levofloxacin, doxycycline, venlafaxine, allopurinol, isosorbide mononitrate, fosonipril, propanolol, promethazine, captopril, fluvastatin, cimetidine, sumatriptan, nortriptyline, naproxen, calacyclovir, doxepin, amoxicillin, azithromycin, diltiazem, cefprozil, acyclovir, ciprofloxacin, losartan, and pharmaceutically acceptable salt of any said active agents.

29. The process of claim 26 wherein the active agent is selected from the group consisting of reboxetine, clindamycin, (−)-S-3-(3-methylsulfonylphenyl)-N-n-propylpiperidine hydrochloride, sumanirole, pramipexole, and pharmaceutically acceptable salt of any said active agents.

30. The process of claim 26 wherein the hydrophobic polymer is selected from the group consisting of wax, wax-like substance, fatty alcohols, shellac, zein, hydrogenated vegetable oils, water insoluble celluloses, cellulose acetate, polymers of acrylic acid, and polymers of methacrylic acid.

31. The process of claim 26 wherein the hydrophic polymer is ethylcellulose.

32. The process of claim 26 wherein the film coating further comprises a pore former.

33. The process of claim 32 wherein the pore former is selected from the group consisting of lithium carbonate, sodium chloride, sodium bromide, potassium chloride, potassium sulfate, potassium phosphate, sodium acetate, sodium citrate, hydroxypropyl methylcellulose, cellulose ethers and protein-derived materials, polyvinylpyrrolidone, cross-linked polyvinylpyrrolidone, polyethylene oxide, polyethylene glycol, pullulan, dextran, sucrose, glucose, fructose, mannitol, lactose, mannose, galactose, and sorbitol.

34. The process of claim 32 wherein the pore former is hydroxypropyl methylcellulose.

35. A solid dosage form made by the process of claim 26.

36. An extended release composition, comprising:
a. a matrix core comprising intragranular ethylcellulose and clindamycin granulated and compressed together with extragranular ethylcellulose; and
b. a film coating comprising a hydrophobic polymer, wherein the film coating completely encases the matrix core.

37. The extended release composition of claim 36, wherein the clindamycin is released at a zero-order rate for a period of at least 8 hours after oral administration to a subject.

38. The extended release composition of claim 36, wherein the clindamycin is in a form selected from the group consisting of clindamycin HCl and clindamycin crystalline free base.

39. The extended release composition of claim 36, wherein the clindamycin is clindamycin HCl.

40. The extended release composition of claim 36, wherein the intragranular and extragranular ethylcellulose in the matrix core are present in a total amount from about 15% to about 99% by weight, relative to total weight of the matrix core.

41. The extended release composition of claim 36, wherein the matrix core further comprises a filler.

42. The extended release composition of claim 38, wherein the filler is selected from the group consisting of microcrystalline cellulose, sodium citrate, dicalcium phosphate, colloidal silicon dioxide, starches, lactose, sucrose, glucose, mannitol, silicic acid, alginates, gelatin, polyvinylpyrrolidinone, and acacia.

43. The extended release composition of claim 38-, wherein the matrix core further comprises a lubricant.

44. The extended release composition of claim 40, wherein the lubricant is selected from the group consisting of stearic acid salts, stearic acid, stearate family, sodium stearyl fumarate, solid polyethylene glycols, sodium lauryl sulfate, and mixtures thereof.

45. The extended release composition of claim 36, wherein the lubricant is from about 0.1% to about 3.0%, by weight, of the matrix core.

46. The extended release composition of claim 36, wherein the film coating comprises from about 1% to about 33%, by weight, of the matrix core.

47. The extended release composition of claim 36, wherein the hydrophobic polymer in the film coat is selected from the group consisting of wax, wax-like substance, fatty alcohols, shellac, zein, hydrogenated vegetable oils, water insoluble celluloses, cellulose acetate, polymers of acrylic acid, and polymers of methacrylic acid.

48. The extended release composition of claim 36, wherein the film coating further comprises a pore former.

49. The extended release composition of claim 48, wherein the pore former is selected from the group consisting of lithium carbonate, sodium chloride, sodium bromide, potassium chloride, potassium sulfate, potassium phosphate, sodium acetate, sodium citrate, hydroxypropyl methylcellulose, cellulose ethers and protein-derived materials, polyvinylpyrrolidone, cross-linked polyvinylpyrrolidone, polyethylene oxide, polyethylene glycol, pullulan, dextran, sucrose, glucose, fructose, mannitol, lactose, mannose, galactose, and sorbitol.

50. The extended release composition of claim 48, wherein the pore-former is up to about 50%, by weight, of the film coating.

51. The extended release composition of claim 36i wherein the clindamycin is about 1% to about 85%, by weight, of the matrix core.

52. The extended release composition of claim 36, wherein the hydrophobic polymer in the film coat comprises ethylcellulose.

53. The extended release composition of claim 36, wherein at least 1% of the clindamycin in the composition is released in the colon of the subject, after oral administration thereto.

54. The extended release composition of claim 36, in the form of a tablet.
